(19) United States
(12) Patent Application Publication
(10) Pub. No.: US 2026/0212996 A1
(43) Pub. Date: Jul. 23, 2026

(54) IMAGE PROCESSING APPARATUS, IMAGE PROCESSING SYSTEM, AND IMAGE PROCESSING METHOD

(71) Applicant: CANON MEDICAL SYSTEMS CORPORATION, Otawara-shi (JP)

(72) Inventors: Kazuki UTSUNOMIYA, Nasushiobara (JP); Shiro KATO, Utsunomiya (JP); Yusaku AIZAWA, Utsunomiya (JP)

(73) Assignee: CANON MEDICAL SYSTEMS CORPORATION, Otawara-shi (JP)

(21) Appl. No.: 19/451,068

(22) Filed: Jan. 16, 2026

(30) Foreign Application Priority Data
Jan. 21, 2025 (JP) 2025-008365

(51) Int. Cl. G16H 30/20; G16H 10/60

(52) U.S. Cl. CPC G16H 30/20; G16H 10/60

(57) ABSTRACT

An image processing apparatus of an embodiment includes processing circuitry. The processing circuitry is configured to acquire a subject image by capturing an image of a subject in a hospital, generate text information corresponding to a situation of the subject based on the subject image, transmit the subject image and the text information to a terminal device of a user, and control whether to cause the subject image and the text information to be recorded in a storage.

### CROSS-REFERENCE TO RELATED APPLICATION

The present application claims priority based on Japanese Patent Application No. 2025-008365 filed Jan. 21, 2025, the content of which is incorporated herein by reference.

### FIELD

The embodiments disclosed in this specification and drawings relate to an image processing apparatus, an image processing system, and an image processing method.

### BACKGROUND

In recent years, due to factors such as a shortage of medical staff in medical settings, it has become difficult to ascertain detailed situations within hospitals. Furthermore, when a patient's situation changes, medical professionals are required to record that situation, but if they are unable to find the time to record the situation and put it off, there is a risk that input of necessary detailed situations will be omitted. To address this issue, methods have been proposed, such as using devices to monitor patients within hospitals and simplifying recording using voice input or bedside terminals. Recording a patient's situation requires ascertaining the patient's situation and recording specific explanatory text, but generating such text places a significant burden on medical professionals.

### BRIEF DESCRIPTION OF THE DRAWINGS

FIG. 1 is a configuration diagram showing an example of a medical information processing system S according to an embodiment.

FIG. 2 is a diagram showing an example of input and output of a generative model M according to an embodiment.

FIG. 3 is a functional block diagram showing an example of a medical information processing device 1 according to an embodiment.

FIG. 4A is a diagram showing an example of a patient situation display screen according to an embodiment.

FIG. 4B is a diagram showing another example of a patient situation display screen according to an embodiment.

FIG. 4C is a diagram showing an example of a patient situation display screen (recording operation) according to an embodiment.

FIG. 4D is a diagram showing an example of a patient situation display screen (non-recording operation) according to an embodiment.

FIG. 5 is a diagram showing an example of patient situation data PD according to an embodiment.

FIG. 6 is a diagram showing another example of a patient situation display screen according to an embodiment.

FIG. 7 is a diagram showing a state in which a part of patient situation text PST is selected according to an embodiment.

FIG. 8 is a diagram showing an example of priority reference information PR according to an embodiment.

FIG. 9 is a sequence diagram showing an example of a processing flow of the medical information processing system S according to an embodiment.

FIG. 10 is a diagram showing an example of input and output of a generative AI model according to modified example 1.

FIG. 11 is a diagram showing an example of input and output of a generative AI model according to modified example 2.

### DETAILED DESCRIPTION

Hereinafter, an image processing apparatus, an image processing system, and an image processing method of embodiments will be described with reference to the drawings. The medical information processing system generates text describing a situation of a patient (hereinafter referred to as “patient situation text”) from images (moving images) of the patient captured by a monitoring camera installed in a hospital, for example, and prompts medical staff to take an action (to perform recording/do not perform recording) based on the content of the patient situation text. This allows medical staff to rapidly ascertain the situation of the patient and reduce the burden of recording the situation of the patient. The patient situation text is an example of “text information.” Medical staff are an example of a “user.”

An image processing apparatus of an embodiment includes processing circuitry. The processing circuitry is configured to acquire a subject image by capturing an image of a subject in a hospital, generate text information corresponding to a situation of the subject based on the subject image, transmit the subject image and the text information to a terminal device of a user, and control whether to cause the subject image and the text information to be recorded in a storage.

### System Configuration

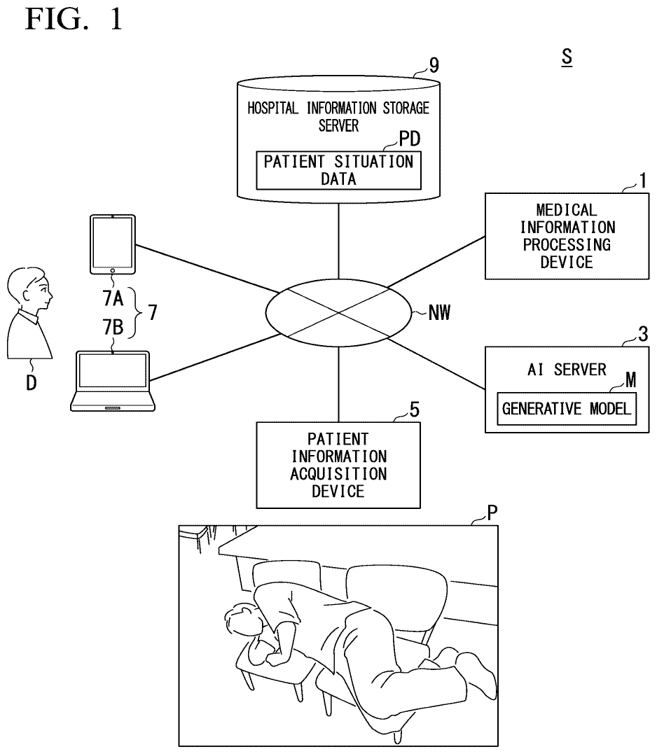

FIG. 1 is a configuration diagram showing an example of a medical information processing system S according to an embodiment. The medical information processing system S includes, for example, a medical information processing device 1 (image processing apparatus), an artificial intelligence (AI) server 3, a patient information acquisition device 5, a terminal device 7, and a hospital information storage server 9. These devices and servers are connected to each other via a communication network NW such that they can communicate.

The communication network NW may refer to any information and communication network that utilizes telecommunications technology. For example, the communication network NW may include telephone communication networks, optical fiber communication networks, cable communication networks, satellite communication networks, and the like in addition to wireless/wired LANs such as a hospital backbone local area network (LAN) and the Internet.

The medical information processing device 1 controls the overall operation of the medical information processing system S. The medical information processing device 1 acquires patient information indicating a situation of a patient in a hospital from the patient information acquisition device 5, generates patient situation text that represents the situation of the patient in text from the acquired patient information, and performs processing to prompt medical staff (e.g., a doctor D, a nurse, etc.) to take an action (perform recording/do not perform recording) depending on the content of the patient situation text. The configuration of the medical information processing device 1 will be described in detail later. A patient is an example of a “subject.” Patient information is an example of “subject information indicating a subject's situation.”

In response to a request from the medical information processing device 1, the AI server 3 generates patient situation text which represents a patient's situation in text from patient information. The AI server 3 generates the text using, for example, a generative model M. The generative model M may be implemented using a large language model (LLM) such as a generative pre-trained transformer (GPT), a pathways language model (PaLM), a large language model meta AI (LLaMA), or Claude. Further, the generative model M may also be implemented using other generative AI such as a variational auto-encoder (VAE) or a generative adversarial network (GAN).

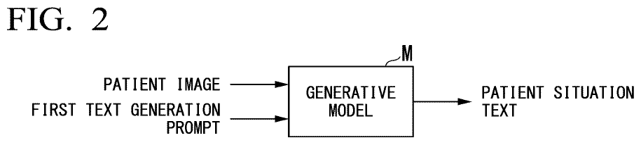

FIG. 2 is a diagram showing an example of input and output of the generative model M according to an embodiment. As shown in FIG. 2, the generation model M outputs patient situation text when an image of a patient (hereinafter referred to as a “patient image”) captured by the patient information acquisition device 5 and a prompt (hereinafter referred to as a “first text generation prompt”) requesting the generation of patient situation text are input. The first text generation prompt includes, for example, “Please explain the situation in this video. Also, is this situation dangerous?” The patient image is an example of a “subject image.”

The patient information acquisition device 5 acquires patient information indicating a patient's situation within the hospital. The patient information acquisition device 5 may be, for example, a camera (monitoring camera), a microphone, or the like installed in any place within the hospital. Patient information includes, for example, an image of the patient captured by the camera (patient image: video) and the voice of the patient acquired by the microphone. FIG. 1 shows an example in which a patient image P of a patient lying in a chair is acquired by the patient information acquisition device 5, which is a camera. Note that the patient information acquisition device 5 may also include communication (e.g., email, telephone audio, etc.) between the patient and medical staff and patient location information (global positioning system (GPS), indoor positioning system). In the following, an example in which the patient information acquisition device 5 is a camera (monitoring camera) will be described. The patient information acquisition device 5 is an example of a “subject information acquisition device.”

The terminal device 7 is a device for referencing various types of information provided by the medical information processing device 1 and inputting various instructions. The terminal device 7 is operated by, for example, medical staff such as a doctor D or a nurse. The terminal device 7 is, for example, a mobile terminal 7A such as a tablet or a smartphone, or a personal computer 7B. The terminal device 7 has, for example, a communication function for performing data communication with other devices, an input interface function for receiving various instructions from medical staff, a display function for displaying various types of information, etc.

The hospital information storage server 9 stores various types of information within the hospital. For example, the hospital information storage server 9 stores patient situation data PD generated by the medical information processing device 1. The hospital information storage server 9 is implemented, for example, using a semiconductor memory device such as a read only memory (ROM), a random access memory (RAM), or a flash memory, a hard disk drive (HDD), and an optical disc. The hospital information storage server 9 is an example of a “storage.”

### Medical Information Processing Device

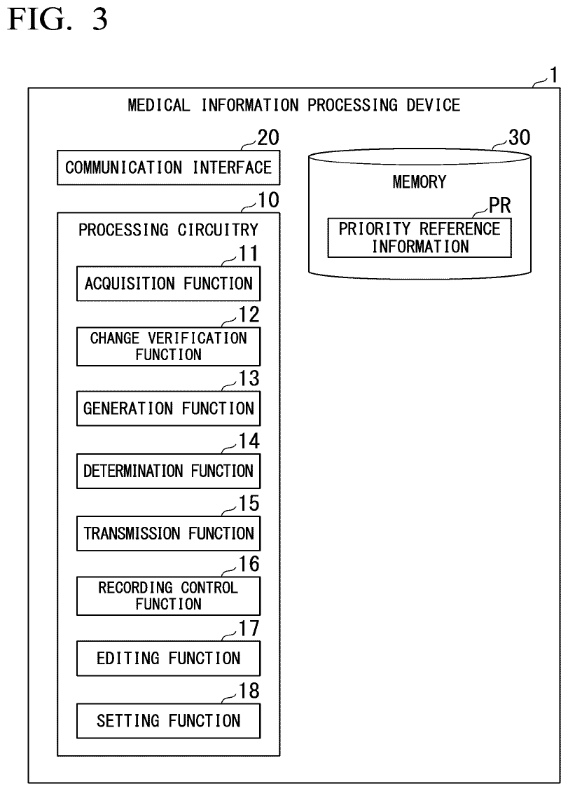

FIG. 3 is a functional block diagram showing an example of the medical information processing device 1 according to an embodiment. The medical information processing device 1 is, for example, a workstation, a server, or the like. The medical information processing device 1 includes, for example, processing circuitry 10, a communication interface 20, and a memory 30. The communication interface 20 communicates with external devices such as the AI server 3, the patient information acquisition device 5, the terminal device 7, and the hospital information storage server 9 via the communication network NW. The communication interface 20 includes a communication interface such as a network interface card (NIC).

The processing circuitry 10 controls the overall operation of the medical information processing device 1. The processing circuitry 10 includes, for example, an acquisition function 11, a change verification function 12, a generation function 13, a determination function 14, a transmission function 15, a recording control function 16, an editing function 17, and a setting function 18. The processing circuitry 10 realizes these functions by, for example, a hardware processor (computer) executing a program stored in the memory 30 (storage circuitry).

The hardware processor refers to circuitry such as a central processing unit (CPU), a graphics processing unit (GPU), an application specific integrated circuit (ASIC), or a programmable logic device (e.g., a simple programmable logic device (SPLD), a complex programmable logic device (CPLD), or a field programmable gate array (FPGA)). Instead of storing a program in the memory 30, the hardware processor may be configured such that the program is directly embedded in the circuitry. In this case, the hardware processor realizes the function by reading and executing the program embedded in the circuitry.

The aforementioned program may be stored in the memory 30 in advance, or may be stored in a non-transitory storage medium such as a DVD or a CD-ROM, and installed into the memory 30 from the non-transitory storage medium when the non-transitory storage medium is inserted into a drive device (not shown) of the medical information processing device 1. The hardware processor is not limited to being configured as a single circuit, and may be configured as a single hardware processor by combining a plurality of independent circuits to realize each function. Furthermore, a plurality of components may be integrated into a single hardware processor to realize each function.

The acquisition function 11 acquires various types of information from external devices via the communication network NW. The acquisition function 11 acquires patient information from the patient information acquisition device 5. The acquisition function 11 also acquires patient situation text from the AI server 3. The acquisition function 11 is an example of an “acquisition unit.”

The change verification function 12 verifies whether a change has occurred in the situation of the patient based on the acquired patient information. The change verification function 12 determines that a change has occurred in the situation of the patient based on a patient image (snap image: video) included in the patient information. For example, if the patient who normally lies in the bed is now lying on the floor in the patient image, the change verification function 12 determines that a change has occurred in the situation of the patient. The change verification function 12 also determines that a change has occurred in the situation of the patient when the patient's position has changed in a time-series patient image of the patient included in the patient information, for example, when the patient lying in the bed falls from the bed. The change verification function 12 may also identify the patient from the patient image. The change verification function 12 is an example of a “change verification unit.”

The generation function 13 generates (in real time) patient situation text corresponding to the situation of the patient on the basis of the patient information. For example, if the change verification function 12 confirms that a change has occurred in the patient, the generation function 13 transmits the patient information and a prompt requesting the generation of patient situation text to the AI server 3, and generates the patient situation text by receiving the patient situation text from the AI server 3. For example, the generation function 13 communicates with the AI server 3 via a dedicated application programming interface (API) provided by the AI server 3. Note that the generation function 13 may be configured to continuously request the patient situation text from the AI server 3 regardless of whether there has been a change in the situation of the patient. The generation function 13 is an example of a “generation unit.” That is, the generation function 13 inputs a patient image (subject image) and a first text generation prompt requesting the generation of patient situation text (text information) to the generative model M (such as a large language model or a multimodal model), and generates the patient situation text (text information) by obtaining the patient situation text (text information) output from the generative model M.

The determination function 14 determines the priority of response to the patient on the basis of the patient situation text. The determination function 14 determines the priority on the basis of results of comparing the patient situation text with priority reference information PR, which includes predetermined priority keywords pre-stored in the memory 30. The determination function 14 is an example of a “determination unit.”

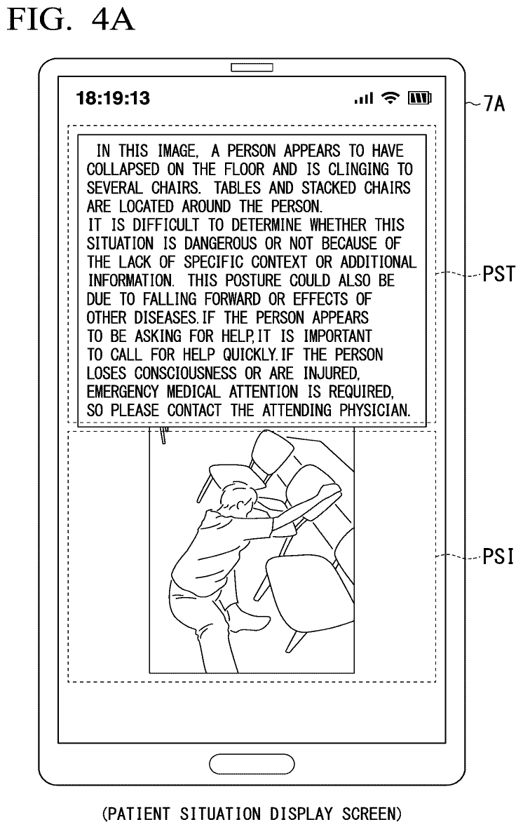

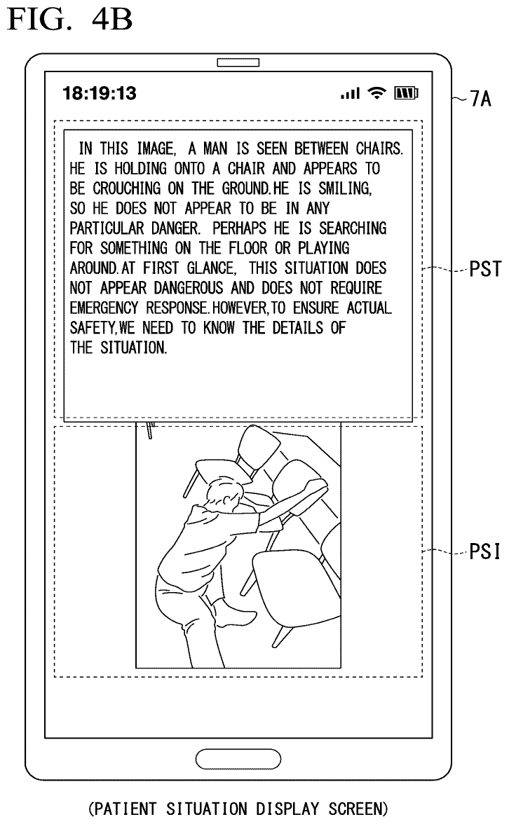

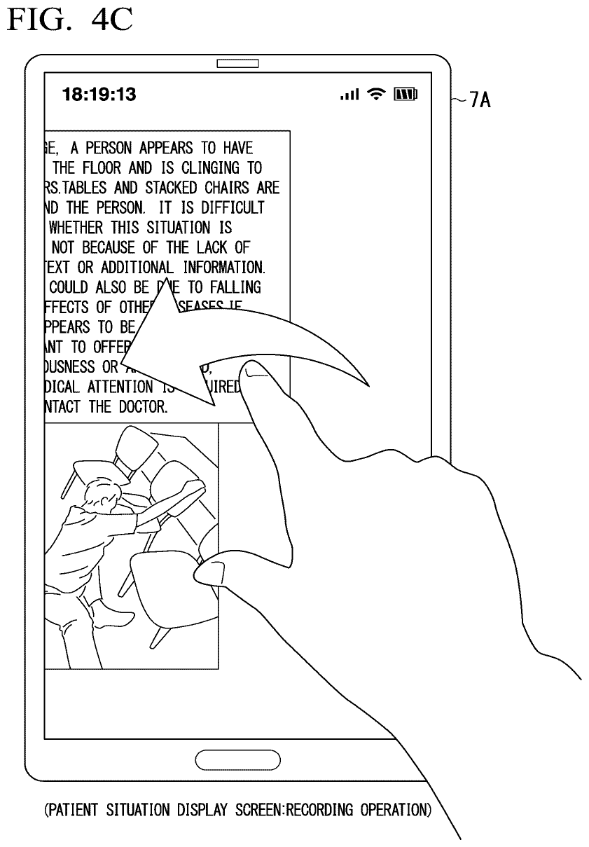

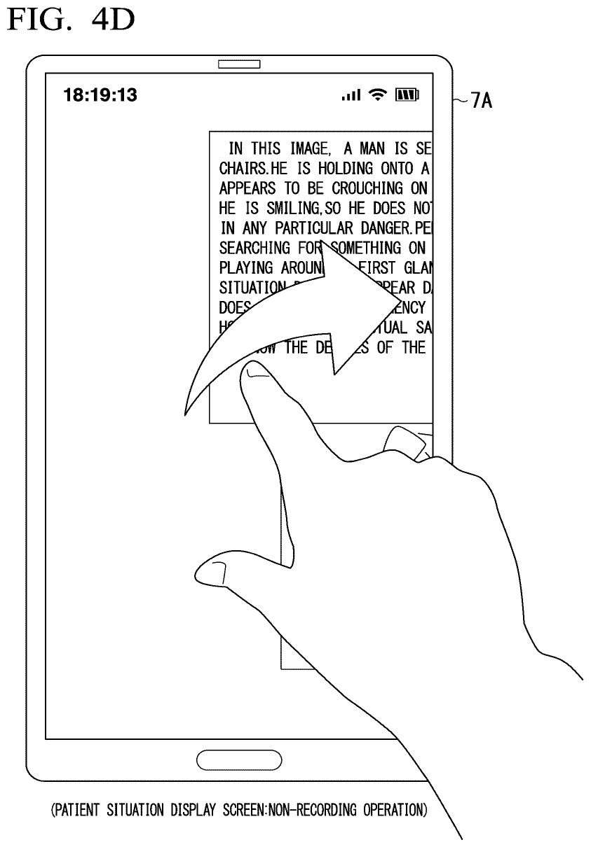

The transmission function 15 transmits an alert notification and patient situation information, including patient information and patient situation text, to the terminal device 7 of medical staff. FIG. 4A is a diagram showing an example of a patient situation display screen displayed on the terminal device 7 (mobile terminal 7A) according to an embodiment. In the example of FIG. 4A, a patient image PSI and patient situation text PST are displayed on the display screen of the mobile terminal 7A. The patient image PSI shows a patient collapsed on the floor. The patient situation text PST includes text corresponding to this situation of the patient: “In this image, a person appears to have collapsed on the floor and is clinging to several chairs . . . ” Medical staff who check such a patient situation display screen can quickly understand that the patient is in a dangerous situation (and that action is required). The transmission function 15 is an example of a “transmission unit.”

FIG. 4B shows another example of a patient situation display screen displayed on the terminal device 7 (mobile terminal 7A) according to an embodiment. In the example of FIG. 4B, a patient image PSI and patient situation text PST are displayed on the display screen of the mobile terminal 7A. The patient image PSI shows a patient collapsed on the floor but with a smiling expression. The patient situation text PST includes text corresponding to this situation of the patient: “In this image, a man is seen between chairs. He is holding onto a chair and appears to be crouching on the ground. He is smiling, so he does not appear to be in any particular danger . . . ” Medical staff who check this patient situation display screen can quickly determine that the patient is not in a dangerous situation (no action is required).

Note that if the priority determined by the determination function 14 is low (if the priority is lower than a predetermined threshold, for example, if the priority is “low”), the transmission function 15 may not transmit an alert notification and patient situation information to the terminal device 7 of medical staff. Whether to perform an alert notification (for example, whether to transmit an alert notification with a “low” priority) can be set by medical staff by operating the input interface of the terminal device 7. This reduces the number of alert notifications and reduces the burden on medical staff such as doctors D and nurses (such as checking notifications). That is, the transmission function 15 may control whether to transmit patient information (subject information) and patient situation text (text information) to the terminal device 7 on the basis of the priority. If the priority is lower than a predetermined threshold, the transmission function 15 may not transmit the patient information (subject information) and patient situation text (text information) to the terminal device 7.

The recording control function 16 controls whether to record the patient information and patient situation text in the hospital information storage server 9 on the basis of the priority determined by the determination function 14. The recording control function 16 controls whether to record the patient information and patient situation text in the hospital information storage server 9 depending on the operation of medical staff performed on the input interface of the terminal device 7 (results of determination of the medical staff). The recording control function 16 is an example of a “recording control unit.”

The recording control function 16 records the patient information and patient situation text in the hospital information storage server 9 when the medical staff operates the input interface of the terminal device 7 to instruct recording. FIG. 4C is a diagram showing an example of a patient situation display screen (recording operation) according to an embodiment. In this example, a recording operation is performed by the medical professional sliding (swiping) the screen of the terminal device 7 (mobile terminal 7A) in a first direction (e.g., left direction). The recording control function 16 causes the hospital information storage server 9 to record the patient information and the patient situation text on the basis of operation information transmitted from the mobile terminal 7A in response to this recording operation.

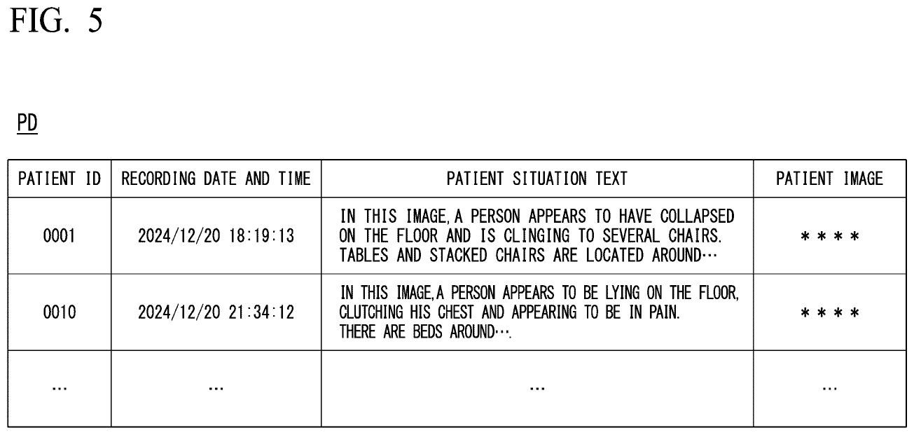

FIG. 5 is a diagram showing an example of patient situation data PD recorded on the hospital information storage server 9 according to an embodiment. In the patient situation data PD, a patient ID for identifying the patient, the recording date and time, patient situation text, and a patient image (patient information) are recorded in association with each other.

When medical staff performs a non-recording operation (deletion operation) to instruct deletion using the input interface of the terminal device 7, the recording control function 16 deletes the patient information and patient situation text without recording the same in the hospital information storage server 9. FIG. 4D is a diagram showing an example of a patient situation display screen (non-recording operation) according to an embodiment. In this example, a recording operation is performed by medical staff sliding (swiping) the screen of the terminal device 7 (mobile terminal 7A) in a second direction (e.g., right direction). The recording control function 16 deletes the patient information and patient situation text from the hospital information storage server 9 without recording the same on the basis of operation information transmitted from the mobile terminal 7A in response to this non-recording operation.

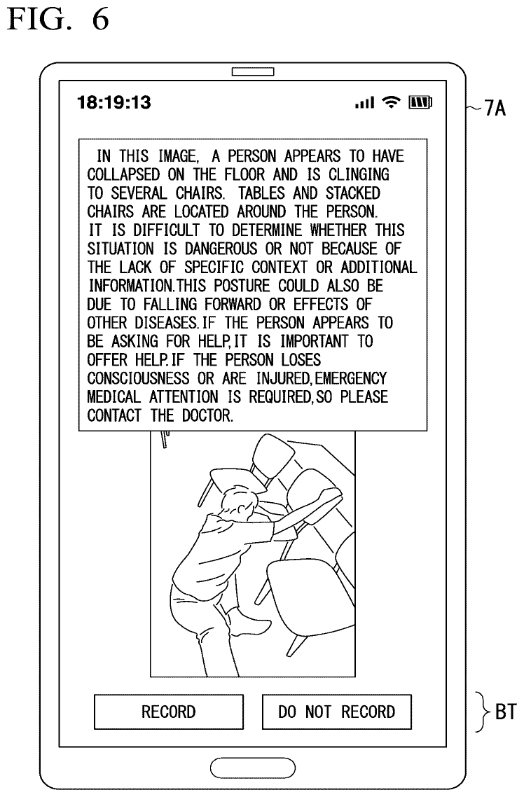

The recording operation and the non-recording operation are not limited to the sliding (swiping) operations described above, and other operations can also be used. FIG. 6 is a diagram showing another example of a patient situation display screen according to an embodiment. This patient situation display screen has a “Record/Do not record” button BT. By pressing this button BT, medical staff can input the result of determination of whether to record or not to record. Alternatively, medical staff may input the result of determination of whether to record or not to record by a voice command.

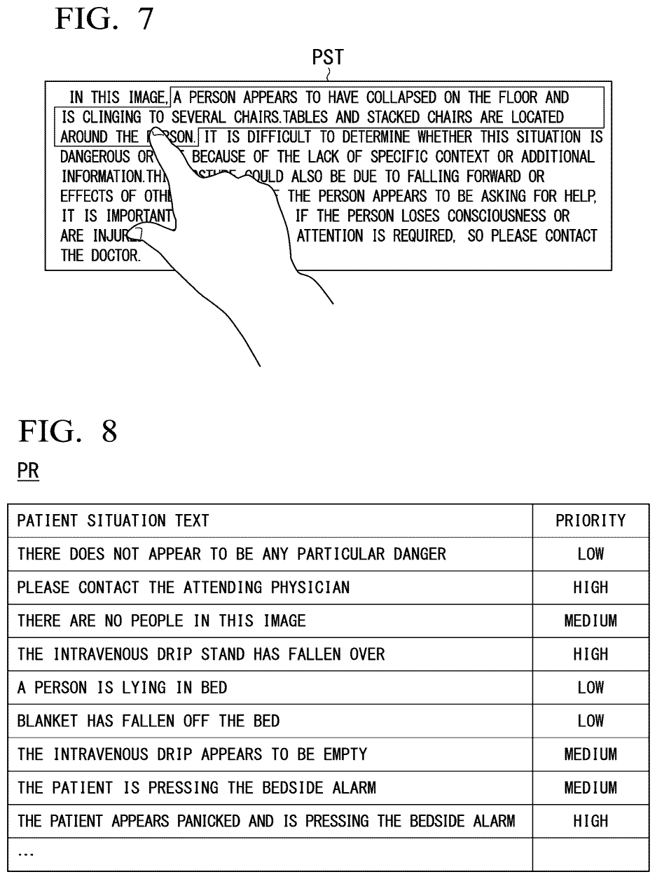

The editing function 17 edits patient information and patient situation text to be recorded in response to the operation performed by medical staff on the input interface of the terminal device 7. For example, the editing function 17 trims patient images (moving images) and edits patient situation text. FIG. 7 is a diagram showing a state in which a part of patient situation text PST is selected according to an embodiment. This example shows a state in which a part of the patient situation text PST, “A person appears to have collapsed on the floor and is clinging to several chairs. Tables and stacked chairs are located around the person.”, is selected (extracted) by a tapping operation.

Alternatively, the editing function 17 may use the functions of the generative AI of the AI server 3 to edit the patient information and patient situation text. For example, the editing function 17 may request that the generative AI of the AI server 3 summarize or translate the patient situation text.

The setting function 18 performs settings with respect to transmission of alert notifications and patient situation information to the terminal device 7. For example, the setting function 18 sets whether to transmit an alert notification and patient situation information according to a priority in response to an operation performed by medical staff on the input interface of the terminal device 7. For example, the setting function 18 sets that an alert notification and patient situation information are not transmitted when the priority is low (when the priority is lower than a predetermined threshold, for example, when the priority is “low”).

The memory 30 stores various types of information. The memory 30 may be realized by, for example, a semiconductor memory element such as a RAM or a flash memory, a hard disk, or an optical disc. These non-transitory storage media may be realized by other storage devices connected via the communication network NW, such as a network attached storage (NAS) or an external storage server device. The memory 30 may also include other non-transitory storage media, such as a ROM and a register. The memory 30 stores, for example, the priority reference information PR and the like. The memory 30 also stores programs, parameter data, and other data used by the processing circuitry 10.

FIG. 8 shows an example of the priority reference information PR according to an embodiment. In the priority reference information PR, patient situation text, which is a specific priority keyword, and a priority are registered in association with each other. In the priority reference information PR, patient situation text “There does not appear to be any particular danger” with a priority of “Low” as an example of a low priority are registered in association with each other. Further, in the priority reference information PR, patient situation text “Please contact the attending physician” and a priority of “High” are registered in association with each other as an example of a high priority.

### Processing Flow

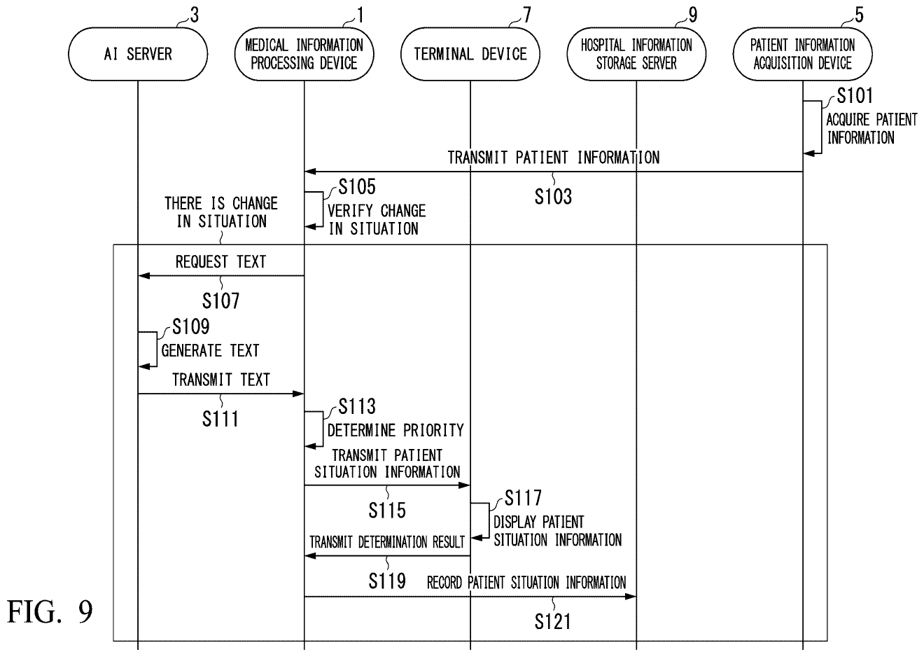

Next, processing in the medical information processing system S will be described. FIG. 9 is a sequence diagram showing an example of a processing flow of the medical information processing system S according to an embodiment.

First, the patient information acquisition device 5 (monitoring camera) installed in any place in the hospital continuously captures images of a patient in the hospital and acquires patient information (patient images) (step S101). The patient information acquisition device 5 continuously transmits the acquired patient information (patient images) to the medical information processing device 1 (step S103).

Next, the change verification function 12 of the medical information processing device 1 verifies whether a change has occurred in the situation of the patient on the basis of a patient image transmitted from the patient information acquisition device 5 (step S105). For example, the change verification function 12 determines that a change has occurred in the situation of the patient when the patient's position has changed, such as when the patient lying in the bed falls off the bed, in time-series patient image (moving image). If it is determined that no change has occurred in the situation of the patient, the medical information processing device 1 does not perform any further processing on this patient image, and repeats the same processing on the next patient information (patient image) transmitted from the medical information processing device 1.

If it is determined that a change has occurred in the situation of the patient, the generation function 13 transmits the patient image and a prompt (first text generation prompt) requesting the generation of patient situation text to the AI server 3, requesting the patient situation text (step S107). For example, the generation function 13 requests patient information text from the AI server 3 via a dedicated API provided by the AI server 3.

Next, in response to the request from the medical information processing device 1, the AI server 3 generates patient situation text that represents the situation of the patient from the patient image (step S109). The AI server 3 generates the patient situation text using, for example, the generative model M. The AI server 3 transmits the generated patient situation text to the medical information processing device 1 (step S111).

Next, the determination function 14 of the medical information processing device 1 determines a priority of response to the patient on the basis of the patient situation text transmitted from the AI server 3 (step S113). For example, the determination function 14 determines the priority on the basis of results of comparing the patient situation text with the priority reference information PR stored in the memory 30. For example, if a specific priority keyword registered in the priority reference information PR is included in the patient situation text, the determination function 14 determines a priority associated with the specific priority keyword registered in the priority reference information PR as the priority of the patient situation text.

Next, on the basis of the determined priority, the transmission function 15 transmits an alert notification and patient situation information including the patient information and the patient situation text to the terminal device 7 of medical staff (step S115). As a result, the terminal device 7 displays a patient situation display screen (e.g., FIG. 4A) using the patient situation information transmitted from the medical information processing device 1 (step S117).

After checking the patient situation display screen, the medical staff operates the input interface of the terminal device 7 to input a result of determining of whether to record the patient situation information (e.g., record nursing). For example, the medical staff inputs a result of determination by performing a recording operation as shown in FIG. 4C or a non-recording operation (delete operation) as shown in FIG. 4D. The terminal device 7 then transmits the input determination result to the medical information processing device 1 (step S119).

Next, if the determination result indicates recording, the recording control function 16 records the patient situation information in the hospital information storage server 9 (step S121). On the other hand, if the determination result indicates non-recording (deletion), the recording control function 16 deletes the patient situation information without recording the same. Accordingly, the series of processing in this sequence diagram end.

The medical information processing system S of the present embodiment described above enables rapid understanding of situations within hospitals that are difficult to monitor due to a small number of staff and reduces the burden of recording those situations. For example, medical staff can record or delete information with simple actions such as a registering operation or an unregistering operation. Furthermore, information on medical situations that was not previously recorded (such as information on what happened where within hospitals) can be recorded without manual intervention, increasing the amount of medical information that can be used. Furthermore, since medical staff can intuitively ascertain the state of the site where an alert is occurring, they can receive support on what to do next and make quick decisions. Furthermore, by setting the system to issue alerts only in truly critical situations, unnecessary alerts can be filtered out and communication resources can be secured. Furthermore, by storing, not all of the patient images and patient situation texts, but only the necessary patient images and patient situation texts, into a storage device, it enables the memory reduction of the storage device. The medical information processing system S of the present embodiment can also be used to understand and record situations of patients in operating rooms and waiting rooms, and situations of care recipients in nursing facilities.

### Modified Example 1

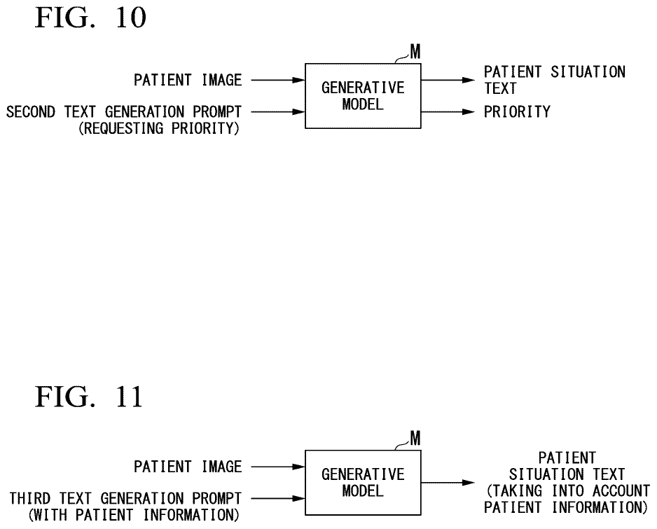

Although the determination function 14 determines the priority of response to the patient on the basis of the patient situation text transmitted from the AI server 3 in the above embodiment, the priority determination may also be performed by the AI server 3. FIG. 10 shows an example of input and output of the generative model M according to modified example 1. When a patient image and a preset prompt (second text generation prompt) requesting the generation of patient situation text and a priority determination are input, the generative model M outputs patient situation text and a priority. The second text generation prompt includes, for example, “Please explain the situation in this video. Also, is this situation dangerous? Please determine the priority.”

That is, the generation function 13 inputs a patient image (subject image) and the second text generation prompt requesting the generation of patient situation text (text information) and the determination of a priority of response to the patient (subject) to the generative model M (large language model), and obtains the patient situation text (text information) and the priority output from the generative model M (large language model).

### Modified Example 2

Although the generation function 13 generates patient situation text on the basis of a patient image and the like in the above embodiment, the patient situation text may also be generated on the basis of pre-recorded patient information in addition to the patient image. This patient information includes various types of information related to the patient's disease, for example, the patient's disease information, a hospitalization history, and the like. This patient information is obtained, for example, from an electronic medical record or hospitalization management system. By generating patient situation text in comparison with such patient information (disease information), it is possible to identify adverse situations occurring in the patient. Furthermore, by inputting in advance information that should be taken into consideration for the patient, it is possible to identify the occurrence of a situation that requires attention.

FIG. 11 is a diagram showing another example of input and output for the generative model M according to modified example 2. As shown in FIG. 11, when a patient image and a prompt (third text generation prompt) requesting the generation of patient situation text dynamically generated in accordance with patient information (disease information) are input, the generative model M outputs patient situation text that takes into account the patient information. The third text generation prompt includes, for example, “Please describe the situation in this video. Also, is this situation dangerous for a patient with ∘∘ disease?,” “Please describe the situation in this video. Also, does this situation correspond to the following situations? 1. ∘∘∘ (a state that requires attention that has been entered in advance), 2. ∘∘∘, 3. ∘∘∘ . . . ,” etc.

That is, the acquisition function 11 further acquires disease information on the patient (subject), and the generation function 13 inputs the patient image (subject image) and the third text generation prompt requesting the generation of patient situation text (text information) dynamically generated in accordance with the disease information to the generative model M (large language model), and generates the patient situation text (text information) by acquiring the patient situation text (text information) output from the generative model M (large language model).

Note that whether to transmit an alert notification and patient situation information to the terminal device 7 may be set depending on the patient information (disease information, disease level). For example, in the case of a patient with a severe disease, the transmission function 15 may transmit an alert notification and patient situation information to the terminal device 7 of medical staff regardless of priority. In addition, whether to transmit an alert notification and patient situation information to the terminal device 7 may be set depending on patient's attributes (sex, age, personality, etc.). By using a machine learning model generated by learning the relationship between patient images and patient information (disease information) and the effectiveness of actual alerts (whether an alert was necessary) using machine learning technology, whether to transmit an alert notification and patient situation information to the terminal device 7 may be set.

Note that, although the medical information processing device 1 controls the overall operation of the medical information processing system S in the above embodiment, some or all of the functions of the medical information processing device 1 may be realized in the terminal device 7. In this case, the terminal device 7 or a combination of the terminal device 7 and the medical information processing device 1 is an example of an “image processing apparatus.” Furthermore, the functions of the AI server 3 may be incorporated into at least one of the medical information processing device 1 and the terminal device 7.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image processing apparatus comprising processing circuitry configured to:
acquire a subject image by capturing an image of a subject in a hospital;
generate text information corresponding to a situation of the subject based on the subject image;
transmit the subject image and the text information to a terminal device of a user; and
control whether to cause the subject image and the text information to be recorded in a storage.

2. The image processing apparatus according to claim 1, wherein the processing circuitry controls whether to cause the subject image and the text information to be recorded in the storage in response to an operation of the user performed on the terminal device.

3. The image processing apparatus according to claim 2, wherein the processing circuitry controls the subject image and the text information to be recorded in the storage in the case where the user operates the terminal device to instruct recording.

4. The image processing apparatus according to claim 2, wherein the processing circuitry does not cause the subject image and the text information to be recorded in the storage in the case where the user operates the terminal device to instruct deletion.

5. The image processing apparatus according to claim 1, wherein the processing circuitry further determines a priority based on the text information, and controls whether to cause the subject image and the text information to be recorded in the storage based on the priority.

6. The image processing apparatus according to claim 5, wherein the processing circuitry determines the priority based on a result of comparison between the text information and priority reference information including pre-registered priority keywords.

7. The image processing apparatus according to claim 5, wherein the processing circuitry determines the priority using a large language model.

8. The image processing apparatus according to claim 1, wherein the processing circuitry further determines a priority based on the text information, and controls whether to transmit the subject image and the text information to the terminal device based on the priority.

9. The image processing apparatus according to claim 8, wherein the processing circuitry does not transmit the subject image and the text information to the terminal device if the priority is lower than a predetermined threshold.

10. The image processing apparatus according to claim 1, wherein the processing circuitry generates the text information in real time and transmits the text information generated in real time to the terminal device.

11. The image processing apparatus according to claim 1, wherein the processing circuitry generates the text information by inputting the subject image and a first text generation prompt requesting generation of the text information to a large language model and acquiring the text information output from the large language model.

12. The image processing apparatus of claim 1, wherein the processing circuitry inputs the subject image and a second text generation prompt requesting generation of the text information and determination of a priority of response to the subject to a large language model, and acquires the text information and the priority output from the large language model.

13. The image processing apparatus of claim 1, wherein the processing circuitry further acquires disease information of the subject, and generates the text information by inputting the subject image and a third text generation prompt requesting generation of the text information dynamically generated in accordance with the disease information to a large language model and acquiring the text information output from the large language model.

14. An image processing system comprising:
the image processing apparatus according to claim 1;
a subject information acquisition device configured to acquire the subject image;
the terminal device configured to display the subject image and the text information; and
the storage in which the subject image and the text information are recorded.

15. An image processing method, using a computer of an image processing apparatus, comprising:
acquiring a subject image by capturing an image of a subject in a hospital;
generating text information corresponding to a situation of the subject based on the subject image;
transmitting the subject image and the text information to a terminal device of a user; and
controlling whether to cause the subject image and the text information to be recorded in a storage.
